(19) United States
(12) Patent Application Publication
(10) Pub. No.: US 2011/0290909 A1
(43) Pub. Date: Dec. 1, 2011

(54) MULTI-COMPONENT INSECT ATTRACTANT

(76) Inventors: Jeff White, North Bend, WA (US); Peter J. Cook, Issaquah, WA (US); John Edward Nkomo, Lynnwood, WA (US); Nathaniel G. Gudz, North Bend, WA (US)

(21) Appl. No.: 13/154,286

(22) Filed: Jun. 6, 2011

Related U.S. Application Data
(63) Continuation-in-part of application No. 11/471,285, filed on Jun. 19, 2006, now abandoned.

(51) Int. Cl. A61L 9/04; A01M 99/00; A01N 25/34; A01N 35/02; A01N 37/10

(52) U.S. Cl. 239/44; 424/84; 424/411

(57) ABSTRACT

Certain embodiments of the present invention employ insect-attracting liquid mixtures, solid mixtures, liquid solutions, and solid solutions that include two or more insect-attracting substances or compounds with different physical characteristics that lead to multi-component plumes comprising component plumes with different radii and multiple chemical gradients. Additional embodiments of the present invention include multi-component insect-attracting substances that effectively target two or more different types of insects that together comprise a target group of insects, and additionally target a particular sex, in certain embodiments, or both sexes, in other embodiments, of the target insects. Many of the insect-attracting-substance embodiments of the present invention, upon controlled vaporization or release, create a multi-component, three-dimensional plume that leads insects to the source of the plume along multiple chemical-concentration gradients. The insect-attracting-substance embodiments of the present invention can be incorporated within lures, traps, and other devices and more complex substances related to insect attraction. In one embodiment of the present invention, a multi-component insect-attracting substance comprises a combination of raspberry ketone and methyl eugenol that is effective, contrary to prior expectations and results, for attracting both oriental fruit flies and melon flies.

### CROSS-REFERENCE TO RELATED APPLICATION

This application is a continuation-in-part of U.S. application Ser. No. 11/471,285, filed Jun. 19, 2006.

### TECHNICAL FIELD

The present invention is related to insect attractants, insect lures, insect traps, and other devices and substances related to attracting insects and, in particular, to multi-component insect-attracting substances containing two or more different insect-attracting component substances with different physical characteristics as well as to insect-attracting devices containing multi-component insect-attracting substances.

### BACKGROUND

Insect-attracting devices and substances have a variety of uses in research, agriculture, pest management, and government-agency-provided services. Insect-attracting substances are used in various types of lures and traps both for monitoring the sizes and dynamics of insect populations as well as for killing insects that are harmful to agriculture, domesticated animals, and human health. Currently available, commercial insect attractants are generally chemical mixtures and solutions containing either a single insect-attracting substance or two or more insect-attracting substances with similar volatilities, dispersal characteristics, and other such physical characteristics. In many applications, the effectiveness of currently available commercial insect attractants either is too low, insufficiently specific for a target type, group, or species of insect, of insufficient general attractiveness to two or more types of insects of a target group of insects, diminishes too quickly through use, or are too expensive for many applications. In certain cases, combinations of attractants, each of which are effective in attracting one particular insect, end up appearing to be relatively less effective, in combination, for attracting one or more of the types of insects than the individual attractants used alone. In other cases, attractants show reasonable effectiveness, when employed in laboratory settings, but lack the volatility and/or other characteristics needed for deployment in the field. However, a combination attractant that could be employed to attract multiple types of insects, would result in significant savings in costs associated with producing, monitoring, and managing insect traps, because only half as many traps would need to be deployed in order to trap the two different types of insects as are needed when single-insect traps are deployed to trap the two different types of insects.

### SUMMARY

Certain embodiments of the present invention employ insect-attracting liquid mixtures, solid mixtures, liquid solutions, and solid solutions that include two or more insect-attracting substances or compounds with different physical characteristics that lead to multi-component plumes comprising component plumes with different radii and multiple chemical gradients. Additional embodiments of the present invention include multi-component insect-attracting substances that effectively target two or more different types of insects that together comprise a target group of insects, and additionally target a particular sex, in certain embodiments, or both sexes, in other embodiments, of the target insects. Many of the insect-attracting-substance embodiments of the present invention, upon controlled vaporization or release, create a multi-component, three-dimensional plume that leads insects to the source of the plume along multiple chemical-concentration gradients. The insect-attracting-substance embodiments of the present invention can be incorporated within lures, traps, and other devices and more complex substances related to insect attraction. In one embodiment of the present invention, a multi-component insect-attracting substance comprises a combination of raspberry ketone and methyl eugenol that is effective, contrary to prior expectations and results, for attracting both Bactrocera dorsalis and B. cucurbitae flies.

### BRIEF DESCRIPTION OF THE DRAWINGS

FIG. 1 illustrates a typical use for insect lures that incorporate and release insect-attracting chemical substances.

FIGS. 2A-C illustrate insect attraction by an insect-attracting chemical substance in the context of the fruit-tree orchard, described with reference to FIG. 1.

FIGS. 3A-B illustrate an extremely effective chemical attractant with a smaller-than-desired effective radius of attraction and capture.

FIGS. 4A-B illustrate a relatively volatile insect-attracting substance with less-than-desired insect-attracting and insect-capturing efficiency.

FIG. 5 illustrates insect-luring features of a flower.

FIGS. 6A-C illustrate multi-component insect-attracting substances that represent embodiments of the present invention.

FIG. 7 illustrates a first method for designing a multi-component insect-attracting substance that represents one embodiment of the present invention.

FIG. 8 is a control-flow diagram illustrating a second, alternative method for designing a multi-component insect-attracting substance that represents one embodiment of the present invention.

FIG. 9 illustrates incorporation of a multi-component insect-attracting substance that represents an embodiment of the present invention within a solid, timed-release lure that represents an additional embodiment of the present invention.

FIG. 10 illustrates four different, exemplary chemical attractants that may be employed in various multi-component insect-attracting-substance embodiments of the present invention directed both to B. dorsalis and B. curcurbitae.

### DETAILED DESCRIPTION

Embodiments of the present invention include insect-attracting mixtures and solutions, and devices and more complex mixtures, solutions, and other substances that incorporate the insect-attracting mixtures and solutions. Insect attractants are widely used by researchers, agriculturists, government agencies, and individuals to detect the presence of harmful insects, to monitor insect populations, and to kill insects harmful to agricultural crops, domesticated animals, and human health. As one example, government agricultural agencies, including both state governments and national governments, monitor fruit crops grown within their jurisdictions in order to be able to certify the fruit as Mediterranean-fruit-fly free, and therefore eligible for export to regions and countries that prohibit importation of fruit from Mediterranean-fruit-fly-infested regions. Insect attractants are used in a wide variety of traps, lures, sprays, atomized solutions and mixtures, insecticides, and in a wide variety of other devices and methods.

In one embodiment, a multi-component insect attractant comprises a combination of methyl eugenol, known to attract Bactrocera dorsalis (oriental fruit fly), and raspberry ketone, a chemical attractant that is chemically related to the insect attractant cue lure. Cue lure is, in fact, raspberry ketone with an acetylated aryl OH group. Cue lure is a known attractant for B. cucurbitae (melon fly). Previous studies had shown that combinations of cue lure and methyl eugenol are relatively ineffective in attracting oriental fruit flies and not significantly better for attracting melon flies. However, as employed in the currently disclosed and currently claimed multi-component insect attractant, the multi-component insect attractant comprising a combination of methyl eugenol and raspberry ketone is significantly more effective for attracting each of these two different types of flies than methyl eugenol, used alone, for attracting oriental fruit flies or cue lure, used alone, for attracting melon flies. Thus, the multi-component insect attractant that represents one embodiment of the present invention represents an unexpectedly and surprisingly effective insect attractant, the components of which having been previously regarded as being ineffective or not more effective for attracting oriental fruit flies and melon flies, respectively.

The current application includes three different research papers related to the methyl-eugenol/raspberry-ketone multi-component insect attractant that represents one embodiment of the present invention. Appendix A includes the article “Effects of Raspberry Ketone on the Mating Success of Male Melon Flies (“Diptera Tephritidae”) by Todd E. Shelly. On page 145 of this article, Shelly states that the study described in this paper “confirms that cue lure and raspberry ketone have similar, short-lasting effects on mating success” and that cue lure “and raspberry ketone also had similar effects of subsequent capture probabilities.” Thus, raspberry ketone and cue lure, which are similar chemically, with cue lure being acetylated raspberry ketone, have similar effects on at least the melon fly.

Appendix B includes the article “Synergistic and Inhibitory Interactions Between Methyl Eugenol and Cue lure Influence Trap Catch of Male Fruit Flies, Bactrocera Dorsalis (Hendel) and B. Cucurbitae (Diptera: Tephritidae)” by Todd Shelly et al. In this paper, discussed in the first paragraph of the discussion section that begins towards the end of page 483, Todd Shelly indicates that a combination of cue lure and methyl eugenol is less attractive to oriental fruit flies than methyl eugenol used alone, while a mixture of methyl eugenol and cue lure is only marginally more attractive to melon flies. Thus, in this study, and in previous studies referred to in this article, a mixture of methyl eugenol and cue lure, which is related to raspberry ketone, is less effective in attracting oriental fruit flies than methyl eugenol used alone and is not significantly more attractive to melon flies than cue lure used alone. Therefore, the effectiveness of the methyl-eugenol/raspberry-ketone multi-component insect attractant that represents one embodiment of the present invention is quite surprising in view of the research discussed and referred to in the paper provided in Appendix B.

Appendix C includes the article “Field Test Assessing the Performance of Farma Tech Mallet-MC Wafers Against Liquid Male Lures” by Todd Shelly. The Farma Tech wafers tested in this study represent a methyl-eugenol/raspberry-ketone multi-component insect attractant that represents one embodiment of the present invention. As shown in this study, this multi-component insect attractant that represents one embodiment of the present invention provides significantly greater attraction of both oriental fruit flies and melon flies than methyl eugenol, used alone, to attract oriental fruit flies and cue lure, used alone, to attract melon flies. Thus, experimental results prove the surprising and unexpected effectiveness of the multi-component insect attractant that represents one embodiment of the present invention.

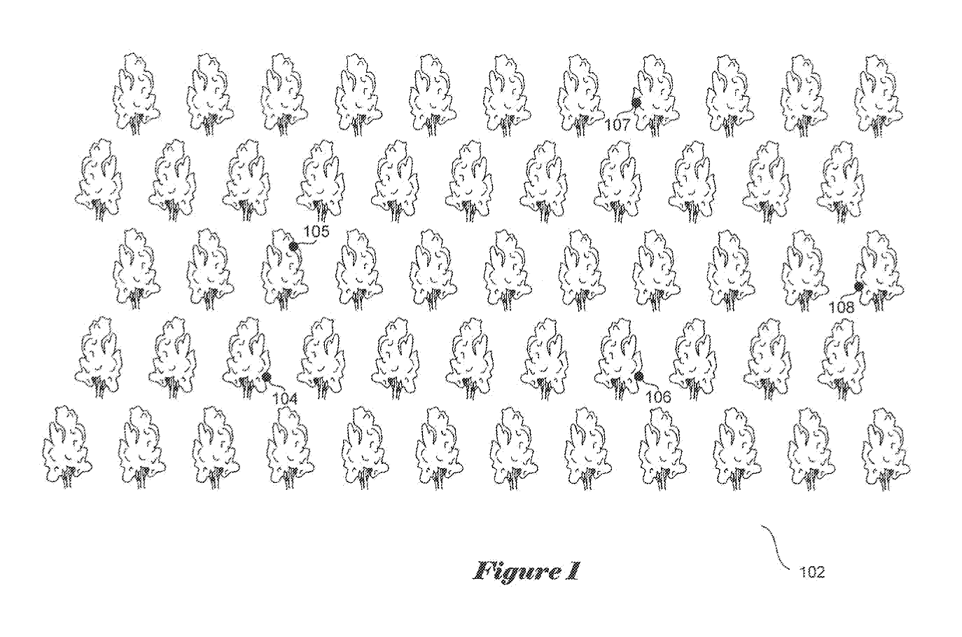

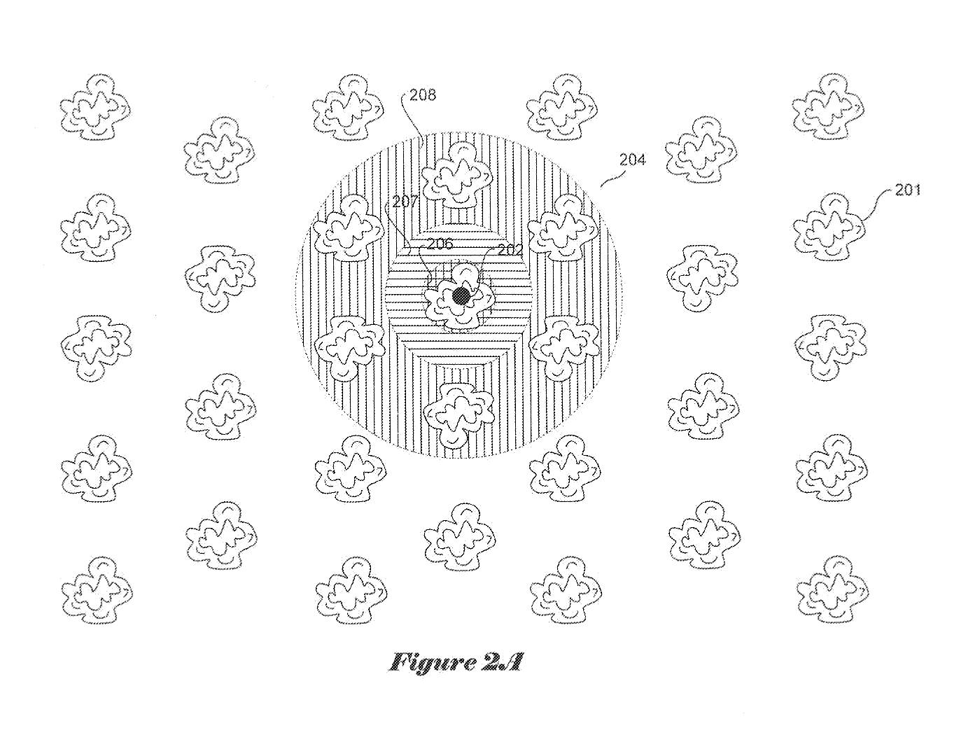

FIG. 1 illustrates a typical use for insect lures that incorporate and release insect-attracting chemical substances. FIG. 1 shows a small portion of a fruit-tree orchard in which a number of insect lures, shown in FIG. 1 as small circular black objects 104-108, have been positioned. In this insect-lure application, a fruit grower uses insect lures to monitor the orchard for the presence of deleterious fruit-fly species. In general, the insect lures are not haphazardly or carelessly positioned, but are instead carefully and regularly distributed throughout the orchard in order to maximize the monitoring efficiency with minimum cost. Cost arises both due to the cost of the insect lures and insect attractants incorporated within, and released by, the insect lures, as well as the cost for routinely accessing the insect lures to identity and count trapped fruit flies, refill the lures with attractant, and check the lures for damage and other problems. For the orchard grower, it is often most desirable to use the fewest possible insect lures, each having the greatest possible radius of effective insect attraction and capture. Using fewer lures minimizes the cost in time and record keeping for accessing and monitoring multiple lures distributed throughout the orchard and the cost in lures and attractants. By contrast, in various agricultural-related and public-health-related research, it may be desirable to use insect lures with smaller effective insect-attraction and insect-capture efficiencies, in order to monitor insect populations within various smaller regions, or, in other words, to monitor insect populations at a finer granularity than an entire orchard or community. Many additional criteria, parameters, and considerations may be involved in developing and effectively using insect lures and insect-attracting substances. For example, the specific attractiveness of an insect-attracting substance may be important in order to specifically target a certain type of insect or even a particular species of insect in order to prevent overloading traps and/or to prevent creating unnatural local high-general-insect populations in the vicinity of traps that may potentially skew population-monitoring results for the particular target insect type or species. The effective capture rate for an insect-attracting substance may be different than the radius of attraction produced when the insect-attracting substance is vaporized or mechanically dispersed, depending on overall attractiveness to insects and on the ability of insects to follow a chemical gradient, generated by vaporizing or mechanically dispersing the insect-attracting substance and subsequent diffusion and transport of the insect-attracting substance into the surrounding environment, to an insect-attracting-substance source within a trap or lure. All of these considerations, parameters, and criteria may need to be evaluated for any given specific application with respect to any particular target insect or group of insects.

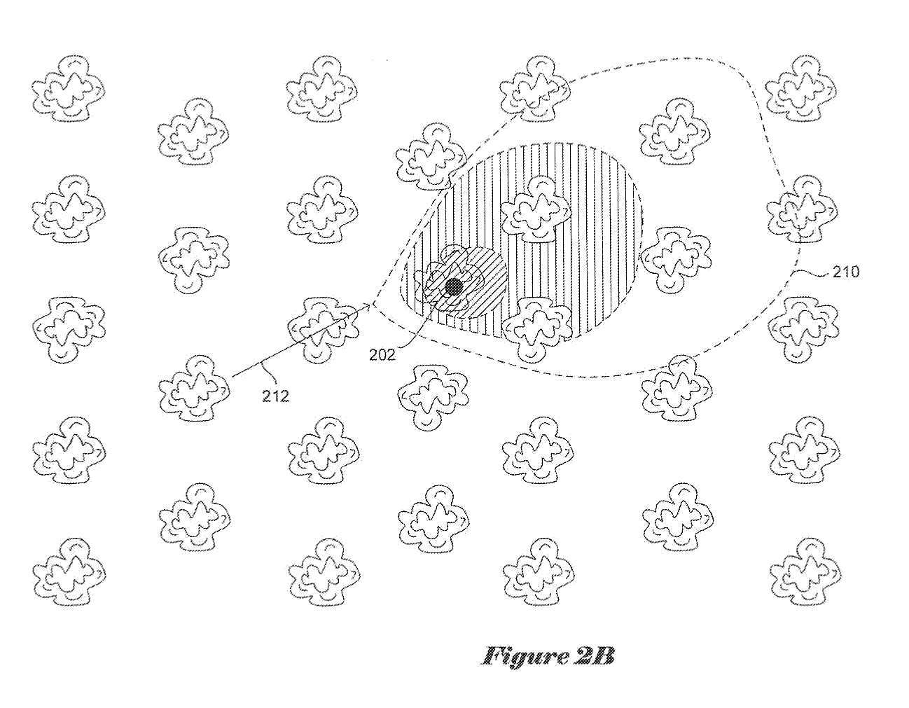

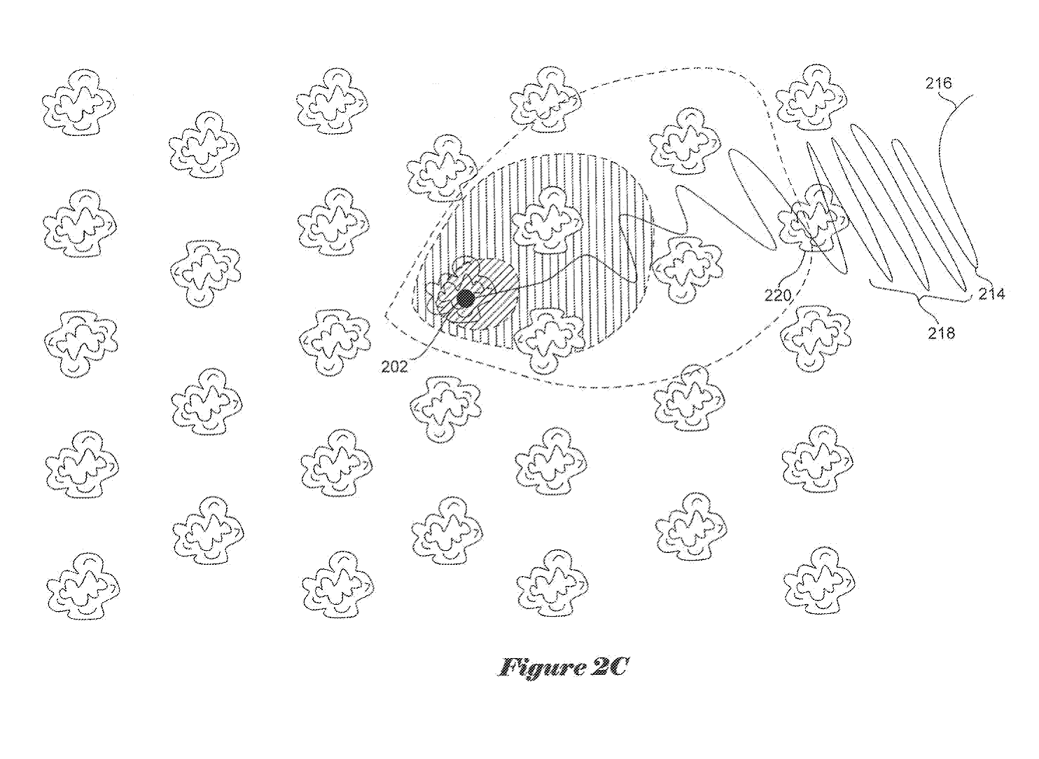

FIGS. 2A-C illustrate insect attraction by an insect-attracting chemical substance in the context of the fruit-tree orchard, described above with reference to FIG. 1. In FIGS. 2A-C, and in subsequent figures, the fruit-tree orchard is viewed from above, with the fruit trees, such as fruit tree 201, arrayed in a regular pattern. As shown in FIG. 2A, on a perfectly windless day, an insect-attracting substance vaporized or released from an insect lure or trap 202 forms a roughly semi-spherical plume of vapor, dispersed droplets, or dispersed particles 204 centered about the trap or lure. In FIG. 2A, the plume is shown having three different concentric, semi-spherical regions 206-208, delineated by different cross hatchings in FIG. 2A. These differently cross-hatched regions are meant to illustrate a chemical gradient, with the concentration of the insect-attracting chemical substance highest in the first region 206 closest to the lure and lowest in the outmost region 208. In fact, in general, the plume of insect-attracting substance varies continuously with distance from the lure, decreasing proportional to the reciprocal of the distance from the lure raised to an exponent generally between two and three, depending on the chemical characteristics of the insect-attracting substance and various environmental characteristics, including atmospheric pressure, humidity, temperature, and other such environmental characteristics. In FIG. 2A, and in subsequent figures, the outer boundary of the plume indicates the boundary of effective attraction, or, in other words, the point past which, in a radial direction from the trap or lure, the concentration of the insect-attracting substance falls below a threshold level needed to attract the target insect type or species. When surface wind is present, the plume is distorted from a semi-spherical shape to a semi-teardrop shape, and may be substantially flattened in the vertical direction. FIG. 2B illustrates the plume of vaporized or dispersed insect-attracting substance when a steady, low-velocity wind is present. As shown in FIG. 2B, the plume becomes semi-teardrop-shaped 210, extending downwind from the lure or trap 202, the wind direction indicated in FIG. 2B by arrow 212. Under varying or blustery wind conditions, insect-attracting-substance plumes may have more irregular and complex shapes, or may be altogether absent due to rapid dispersal and mixing of the insect-attracting substance within a very large volume of air.

FIG. 2C illustrates the path of a flying insect prior to, and after, encountering an insect-attracting-substance plume emanating from a lure or trap. In FIG. 2C, the curved line 214 indicates the flight path of an insect, starting from point 216. Prior to encountering a sufficiently high concentration of the chemical-attractive substance emanating from the lure or trap, the insect may fly in a searching or patrolling pattern 218 characteristic of insects foraging for food sources, seeking mating partners, or seeking nesting locations. When the insect encounters a sufficiently high concentration of insect attractant at the edge of the plume 220, the insect's flight path ideally straightens and become more directed as the insect follows the chemical gradient of insect attractant through regions of increasing insect-attracting-substance concentrations to the lure or trap 202.

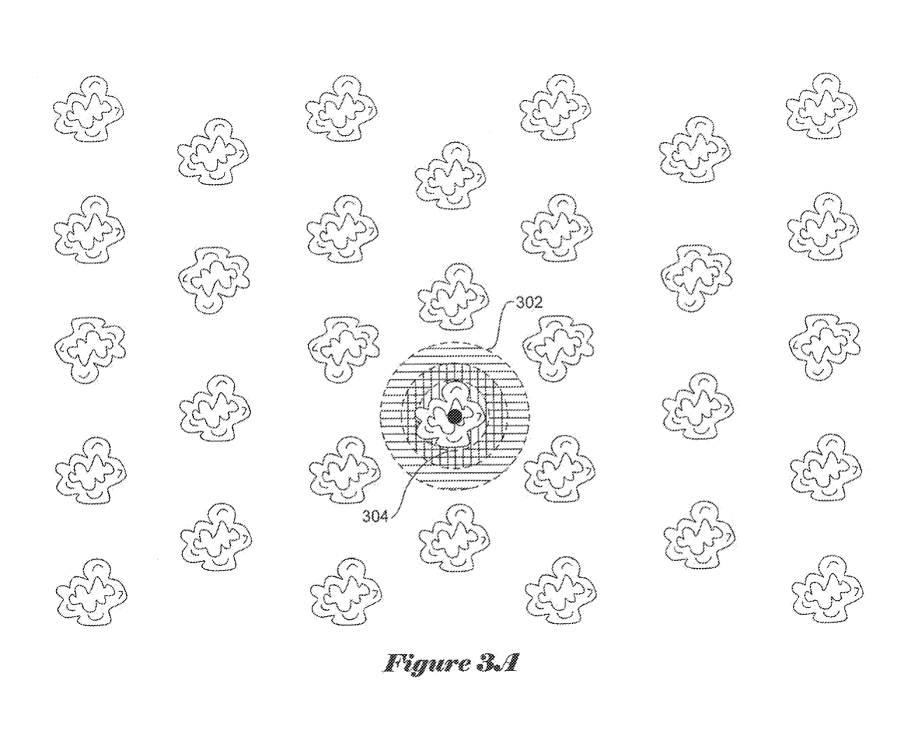

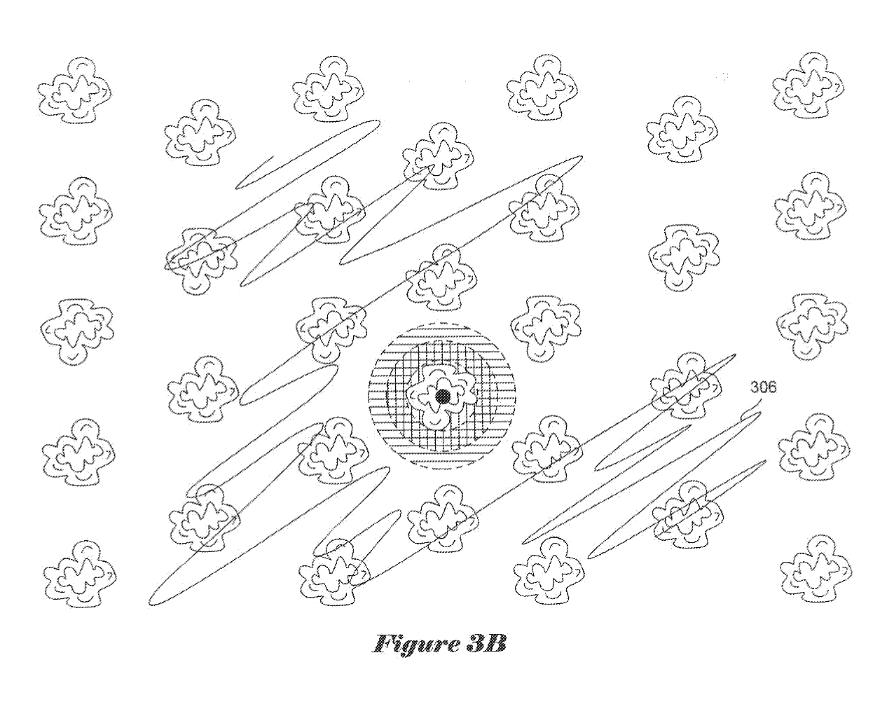

In general, for a given application, such as a particular investigation of the population dynamics of a particular flying insect species, or monitoring of particular types of fruit flies in an agricultural setting, there is a desired or optimal set of characteristics or parameters for an insect lure or insect trap. For example, there may be a desired radius of attraction, a desired capture rate, a desired lure or trap duration of effectiveness, a desired maximum cost, a desired specific attractiveness for a particular species or type, and other such desired or optimal characteristics and parameters. Very often, a particular insect-attracting substance may not meet all of the desired characteristics and parameters. FIGS. 3A-B illustrate an extremely effective chemical attractant with a smaller-than-desired effective radius of attraction and capture. FIG. 3A illustrates the relatively low-volume plume 302 surrounding a lure or trap 304 within the fruit-orchard context of FIGS. 1 and 2A-C produced by a first chemical attractant. Within the plume of effective chemical-attractant concentration 302, the insect-attracting substance may be extremely well targeted to a particular, target insect species, and may be extremely effective at attracting insects of the target species to the lure or trap 304. However, because of the low volatility of the first chemical attractant, the plume of effective concentration 302 may be much smaller than desired. Because of this, as shown in FIG. 3B, an insect typical of the target species, with a relatively broad patrolling pattern 306, may often fail to encounter the plume, and therefore fail to be lured to the trap. This problem may be ameliorated by increasing the amount of chemical-attractant used within traps and lures, increasing the density of lure or trap placement, by using heated traps or traps that mechanically disperse the attractive substance, or by other means, but such ameliorative techniques may increase the cost of the traps above the point where the lures or traps are cost effective for the particular application, and may also be ineffective. For example, even vastly increasing the amount of a chemical attractant with very low volatility may not produce a plume of effective concentration of appreciable and desired size.

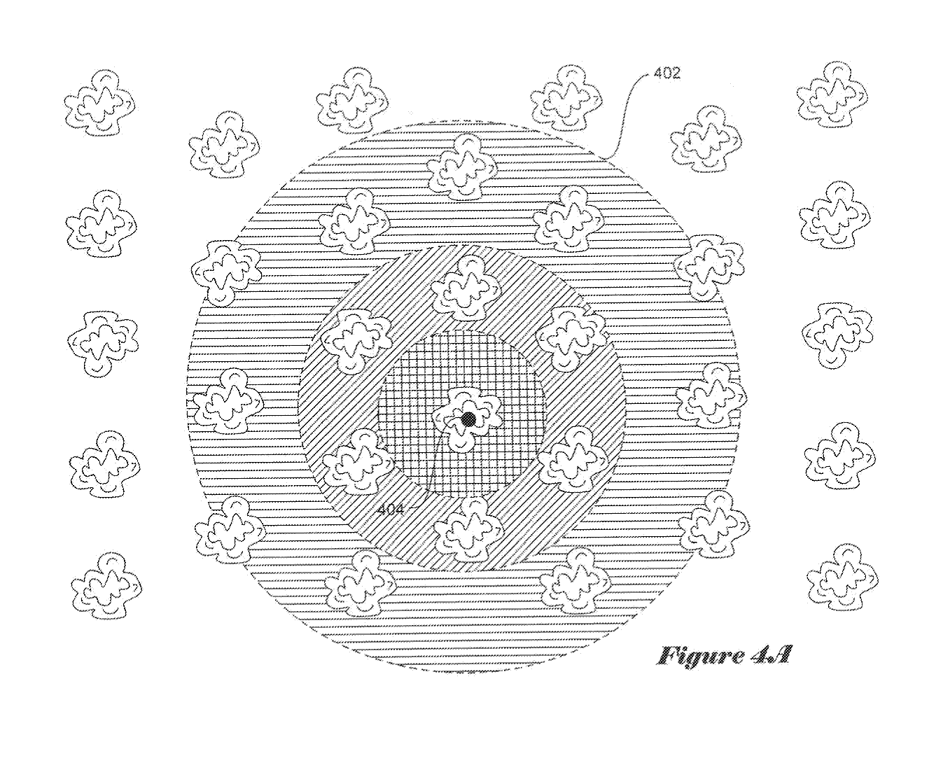

FIGS. 4A-B illustrate a relatively volatile insect-attracting substance with less-than-desired insect-attracting and insect-capturing efficiency. In FIG. 4A, the plume of effective concentration 402 for a second insect-attracting substance emanating from a trap or lure 404 is illustrated in the fruit-orchard context of FIGS. 1, 2A-C, and 3A-C. In this second case, the plume of effective concentration 402 may have the desired radius for the specific application. However, in this case, the more volatile second chemical attractant has much lower overall insect-attracting and insect-capture efficiency. As shown in FIG. 4B, in the case of a trap or lure using the second chemical attractant, the patrolling insect may be efficiently initially attracted by the plume, but the lower insect-attracting efficiency of the second chemical attractant may result in insects entering and exiting the plume without being attracted to the lure or trap.

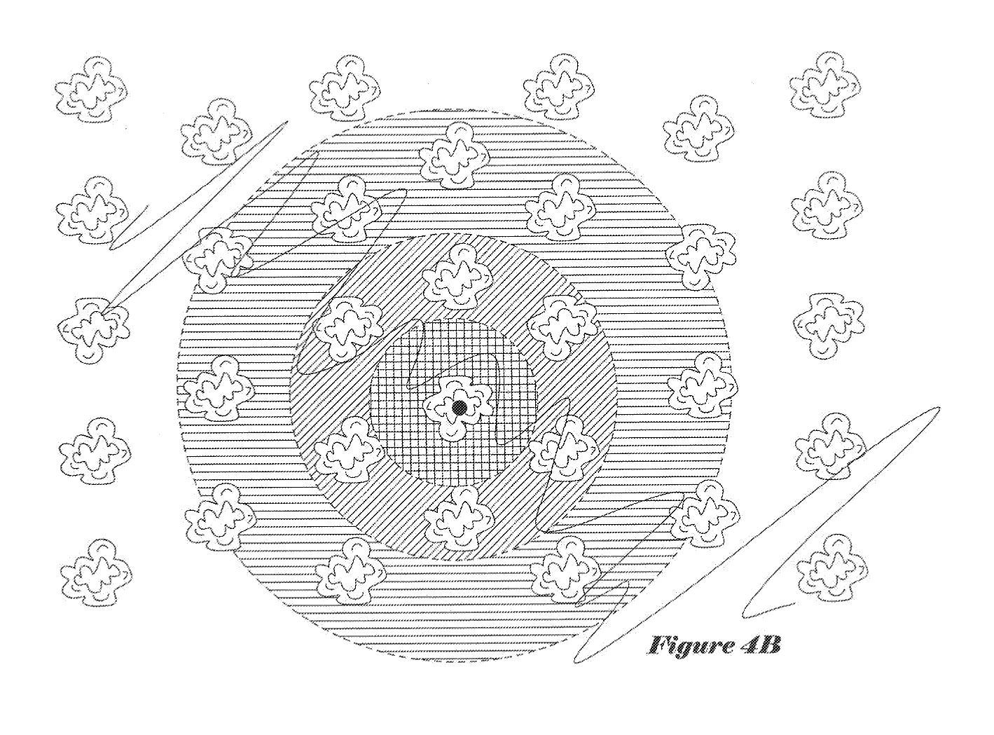

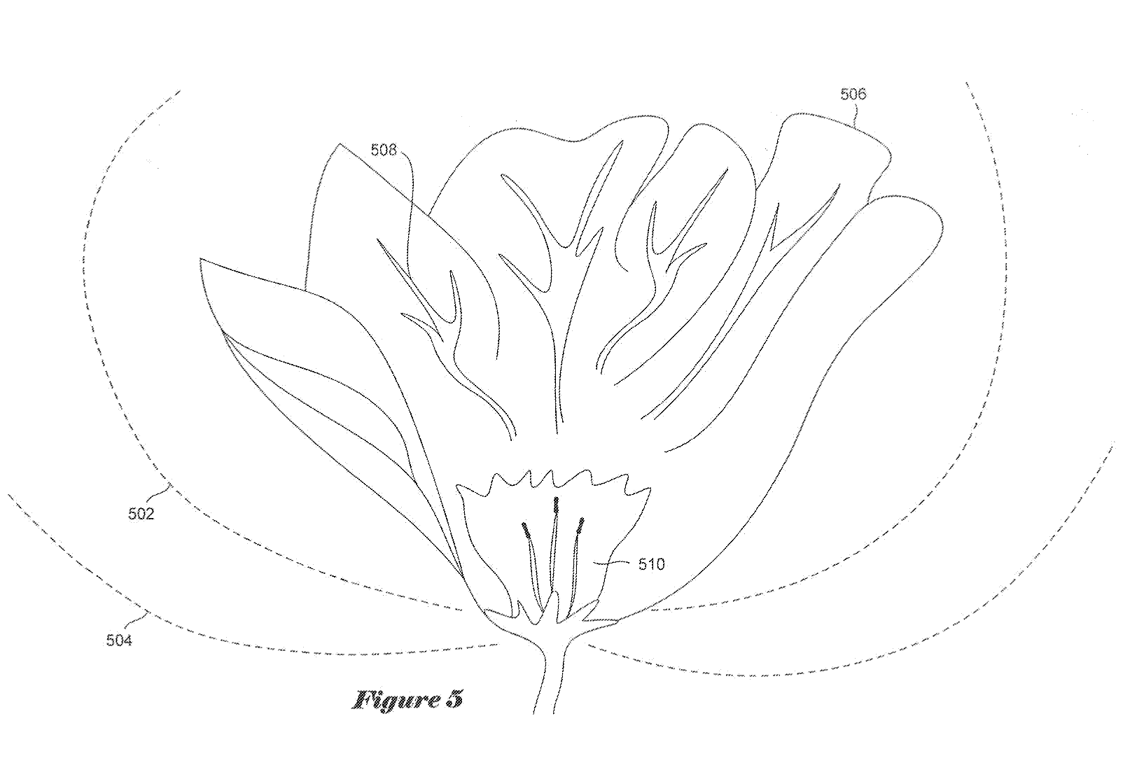

Flowers are one type of naturally occurring, highly evolved, often highly specific, and highly effective insect lure. FIG. 5 illustrates insect-luring features of a flower. Longest-distance attractiveness of a flower may result from chemical attractants emanating from a flower that form a chemical-attractive gradient 504 surrounding the flower. Often, chemical attractants produced by flowers mimic insect pheromones and other naturally occurring insect attractants. In other cases, the chemical attractants are biochemical intermediates used by insects to produce pheromones, food sources, or of other biological use to insects. At shorter distance, the flower may attract insects following the chemical-attractive gradient with visually appealing petals 506, often festooned with elaborate patterns 508 visible at UV wave lengths that lead the insect toward pollen-covered flower features 510. At very close range, within or near the pollen-covered features 510, the flower may employ specially textured surfaces that attract the insect through the insect's tactile senses. Thus, flowers often employ a multi-tiered insect-attraction strategy, with different types of attractants employed at different effective radii of attraction. This multi-tiered insect-attraction strategy may produce multiple, distinct gradients, including chemical gradients, visual gradients, and tactile gradients, to lead insects to a desired region within the flower, where the insect can avail itself of pollen and other nutrients, and the flower can receive pollination from pollen grains loosely bound to the insect. Like flowers, various types of fruit may serve as insect attractants, and may also target olfactory, visual, and tactile senses of target insects.

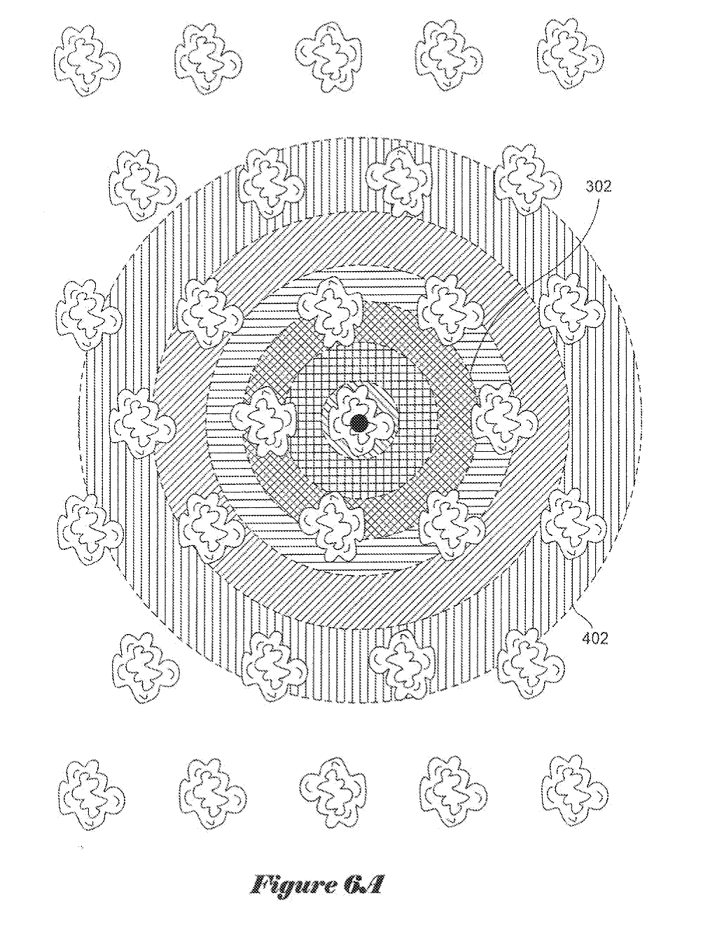

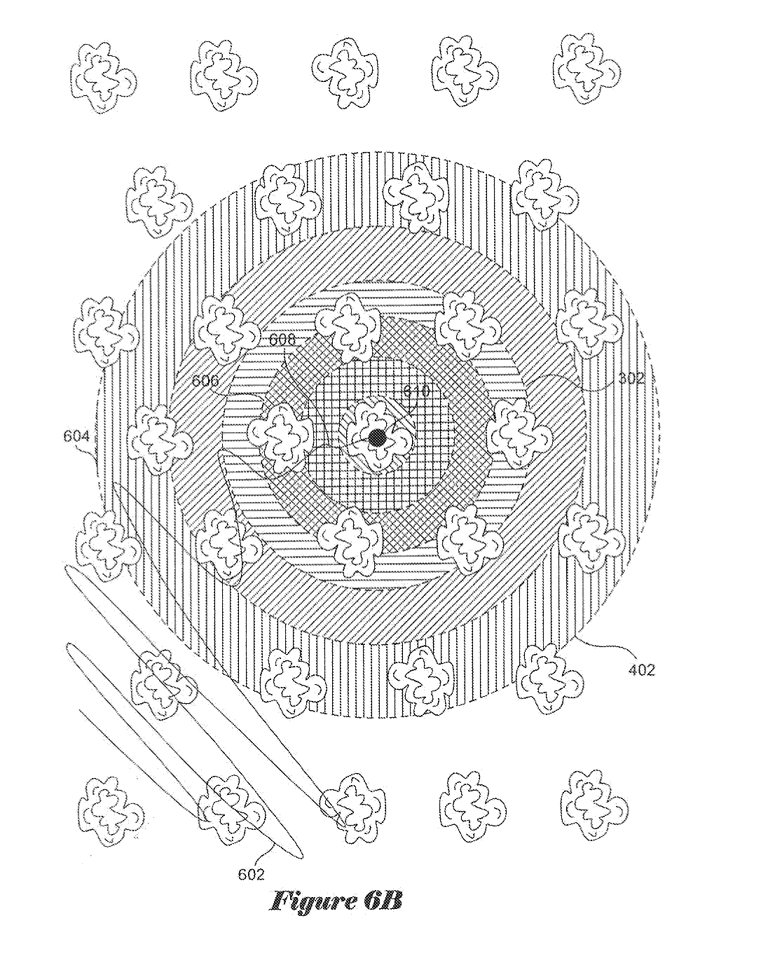

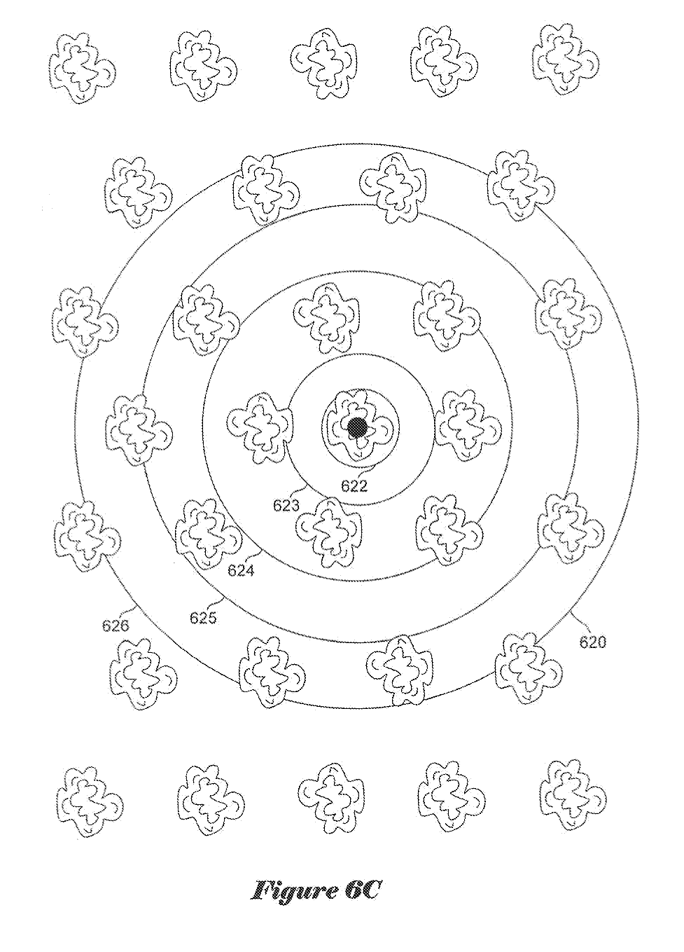

Various embodiments of the present invention address the problems discussed with reference to FIGS. 3A-4B by using multi-tiered insect-attracting gradients, similar to the multi-tiered insect-attraction strategies employed by flowers, although the various embodiments of the present invention employ multiple chemical-attractant gradients, rather than different types of gradients targeting different sensory systems of the insect. FIGS. 6A-C illustrate multi-component insect-attracting substances that represent embodiments of the present invention. Using the example of FIGS. 3A-4C, one embodiment of the present invention, shown in FIG. 6A, combines the first chemical attractant with high insect-attracting effectiveness, but low volatility, discussed with reference to FIGS. 3A-B and the second chemical attractant with lower insect-attraction effectiveness, but higher volatility, discussed with reference to FIGS. 4A-B, to form a multi-component chemical attractant that combines the favorable characteristics of the two discrete chemical attractants to produce a multi-component chemical-attractant plume with multiple chemical-attractant gradients. FIG. 6A shows the smaller-radius effective-concentration component plume 302 generated by the first chemical attractant within the much larger effective-concentration plume 402 produced by the second chemical attractant. As shown in FIG. 6B, a patrolling insect may follow a normal patrolling pattern 602 until encountering the edge 604 of the effective-concentration component plume 402 of the second chemical attractant. The insect's flight path may become more directed as the insect is led towards the trap. When the insect encounters the edge 606 of the effective-concentration component plume 302 of the first chemical attractant, the insect's flight path 608 becomes even more directed as the more effective first chemical attractant leads the insect to the lure or trap 610.

In the first embodiment of the present invention illustrated in FIGS. 6A-B, two different chemical attractants with two different volatilities are employed together to produce a two-component or multi-tiered plume (402 and 302 in FIG. 5A) of effective concentration. In alternative embodiments of the present invention, a larger number of discrete insect-attracting-substance components may be used to produce a larger number of component plumes and insect-attracting-substance gradients within the overall plume. FIG. 6C shows a trap or lure baited with a five-component insect-attracting mixture or solution. The five-component mixture or solution produces a five-way-tiered plume of effective. concentration 620 featuring concentric sub-regions with increasing radii 622-626 generated by the increasingly volatile, five component insect-attracting substances. A multi-component plume with multiple insect-attracting chemical concentration gradients can be achieved by using multiple insect-attracting substances with varying volatilities, or may alternatively be achieved by dispersing or volatilizing the different insect-attracting substances using different methods. In still additional embodiments, the plume components of the overall effective-concentration plume may dynamically change in volume with changing environmental conditions. For example, the range of volatility of one component, depending on temperature, may be far larger than that of a second component, so that the volatility of the first component may be much greater than that of the second component during the day, but much less than that of the second component at night. Such dynamic, multi-tiered insect-attracting-substance effective-concentration plumes may be tailored to behavior patterns of target insects that change with changing environmental conditions, time of day, and other factors.

The multi-component insect-attracting plumes of the present invention are specifically designed to optimize cost, range of attraction, specific attractiveness, and other such characteristics and parameters based on the ability to create multi-component plumes as discussed above. For example, a first, less expensive, more volatile component insect-attracting substance can be used to create an overall radius of effective attraction, while a second, more expensive, less volatile component insect-attracting substance can be used to create a desired, specific attractiveness for insects lured into the sub-plume created by the second component attracting substance by the first component insect-attracting substance. In certain embodiments of the present invention, particular, specific combinations of individual insect-attracting substances may work function synergistically or by a combination effect, in addition to, or instead of, by creating a multi-component plume.

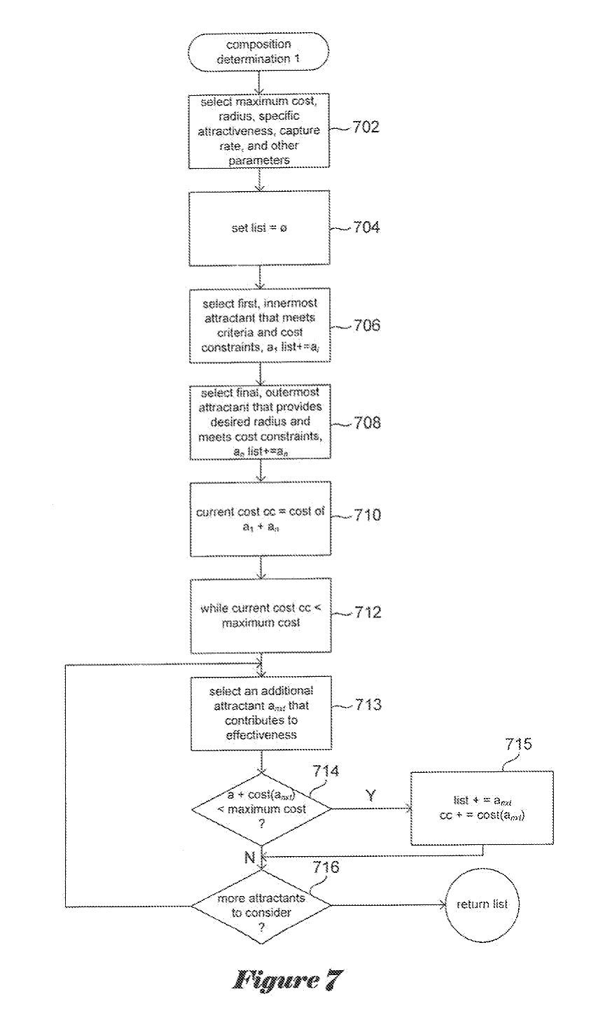

FIG. 7 is a control-flow diagram illustrates a first method for designing a multi-component insect-attracting substance that represents one embodiment of the present invention. In step 702, criteria and parameters that describe the multi-component insect-attracting substance to be designed are selected. As discussed above, these criteria and parameters may include a maximum cost per volume, maximum cost per unit of effective attraction, or maximum cost per lure, an overall radius of effective attraction, a measure of the specific attractiveness of the multi-component substance for a particular target insect type or target insect species, a desired capture rate, a desired duration of effectiveness, and other such parameters and criteria. In step 704, a variable “list” is set to null. The variable “list” used to construct and return a list of chemical attractants that represent the multi-component insect-attractant designed by carrying out the method shown in FIG. 7. In step 706, a first, innermost-component-plume-generating attractant that meets various of the constraints and criteria selected in step 702 is selected and added to the list. For example, the first attractant may be selected in order to guarantee a particular, overall effectiveness or specific attractiveness. Next, in step 704, a second attractant that produces the outermost, largest-diameter component-plume that meets certain of the specified criteria and parameters is selected. For example, the second attractant may be a highly volatile attractant that produces an overall desired radius of the plume of effective concentration. In step 710, the variable “cc,” representing the total cost of the multi-component insect-attracting substance, is initialized to the sum of the costs of the first attractant, selected in step 706, and the second attractant, selected in step 708. Finally, in the while-loop of steps 712-716, additional attractants are selected for the multi-component insect-attracting substance that meets the parameters and constraints specified in step 702 while the total cost of the multi-component insect-attracting substance remains below a specified maximum cost.

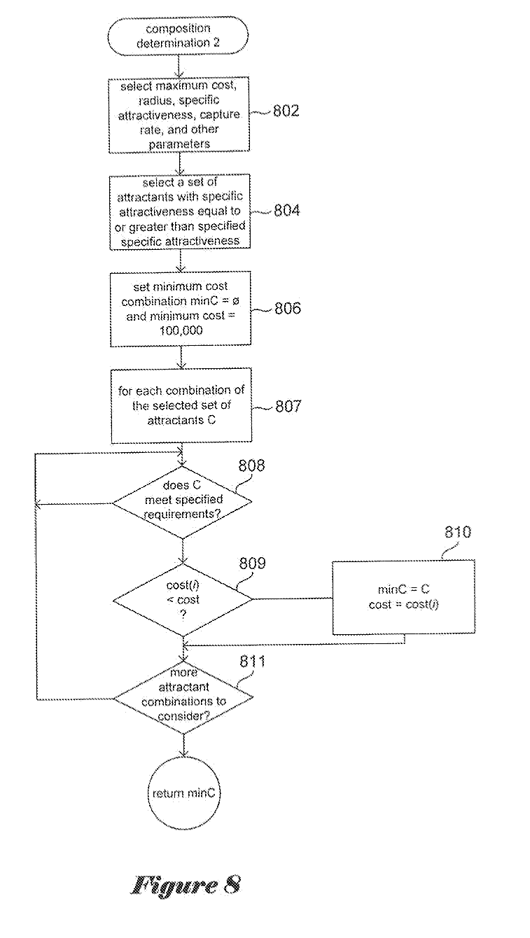

Many alternative methods for designing a multi-component insect-attracting-substance embodiment of the present invention are possible. For example, FIG. 8 is a control-flow diagram illustrating a second, alternative method for designing a multi-component insect-attracting substance that represents one embodiment of the present invention. In step 802, the criteria, constraints, and parameters for the multi-component insect-attracting substance are specified, as in step 702 of FIG. 7. In step 804, a set of different chemical attractants with specific attractiveness for the target insect equal to or greater than a desired specific attractiveness are selected. In step 806, the variable “minC,” representing the most cost-effective considered combination of attractants selected in step 8O4, is initialized to null, and the variable “cost” is set to a large value. Then, in the for-loop of steps 807-811, each possible combination of the attractants selected in step 804 is considered. If the currently considered combination meets the criteria and parameters specified in step 802, as determined in step 808, and has a total cost less than the cost of the most cost-effective combination so far observed, as determined in step 809, then the currently considered combination is selected as the best candidate combination so far observed, in step 810. As a result of the for-loop of steps 807-811, the most cost-effective combination of attractants selected in step 804 that meets the specified criteria and parameters is determined as the result of the multi-component insect-attracting-substance-design method.

In alternative method embodiments of the present invention, additional considerations and criteria may be evaluated during evaluation of each candidate multi-component insect-attracting substance. For example, the radius of attraction of a substance may vary with environmental conditions and overall amount of the substance included in a trap or lure, and these factors may be considered when evaluating various combinations of chemical attractants. In still alternative embodiments, overall effectiveness or specific effectiveness of the multi-component insect-attracting substance may be weighed more heavily than the cost. In summary, the multi-component insect-attracting-substance design methods of the present invention attempt either to identify a combination of insect-attracting-substances that meet specified criteria, parameters, and constraints or to optimize combination of insect-attracting-substances to meet specified criteria and parameters under various constraints. Multi-component insect-attracting-substance design methods may be encoded in software programs or may be carried out manually using printed tables of chemical-attractant properties and characteristics.

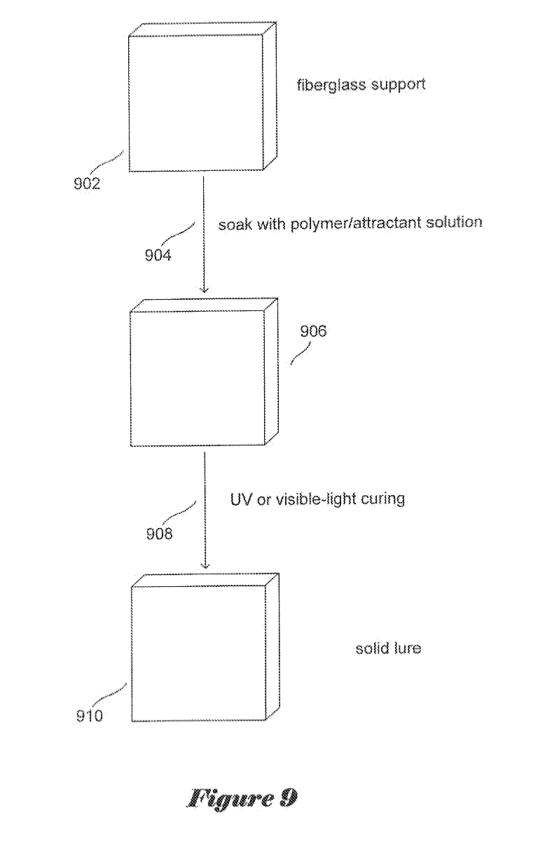

FIG. 9 illustrates incorporation of a multi-component insect-attracting substance that represents an embodiment of the present invention within a solid, timed-release lure that represents an additional embodiment of the present invention. A fiberglass support matrix is first selected 902. In a first step 904 of the method, the fiberglass support is soaked with a liquid mixture of polymer, cross-linking agent, UV initiator, a multi-component insect-attracting substance that represents an embodiment of the present invention, and any additional desired ingredients, such as insecticides for killing lured insects. The first step produces a fiberglass support permeated with polymer, polymer-cross-linking agent, cross-linking initiator, and active ingredients 906. In a second step 908, the nascent lure 906 is exposed to a cross-linking-initiating agent, such as UV light, visible light, heat, pressure, electrical current, or another cross-linking-initiating agent. The second step produces a solid lure 910 in which the multi-component insect-attracting substance that represents an embodiment of the present invention is encased within a cross-linked polymer. The cross-linked polymer may serve as an elastomeric matrix container for the insect-attractants through which chemical-attractant vapor diffuses at a desired rate for controlled release to the environment. In other systems, the cross-linked polymer may be designed to break down, over time, in the environment as a result of exposure to sunlight, moisture, heat, temperature fluctuations, bacteria, or other cross-linked-polymer-degrading agents to slowly and regularly release the multi-component insect-attracting substance that represents an embodiment of the present invention. In still additional embodiments of the present invention, many other types of materials and chemical systems can be devised to retain the chemical attractants for a designed, controlled release. In one solid-lure embodiment of the present invention, the polymer/attractant solution used in step 904 comprises a urethane acrylate oligomer, a trimethyl propane triacrylate monomer cross-linker, any of a variety of UV initiators that produce free radicals upon exposure to UV light, the multi-component insect-attracting substance that represents an embodiment of the present invention, and any additional desired active ingredients, with the active ingredients encapsulated within the resulting elastomeric cross-linked polymer matrix. In alternative embodiments, polyvinyl chloride, silicone, and a wide variety of additional types of polymers may be cross-linked to produce a timed-release matrix container. The multi-component insect-attracting substance may be present in the polymer/attractant solution at 40% to 70% percent by weight, in one class of embodiments, at 10% to 90%, in a second class of embodiments, and from 1% to 99% in a third class of embodiments. Multi-component insect-attracting substances of the present invention may also be used in cotton-wick-based lures and traps, may be used in aerosol sprays, in wax-based traps and lures, in hydrogel-based traps and lures, or may be mixed with a variety of other solutions and substances for various application-specific utilities such as, for example, in emulsified sprayable formulations, attractant glue formulations, and simple applications to cardboard, paper, or paper mache.

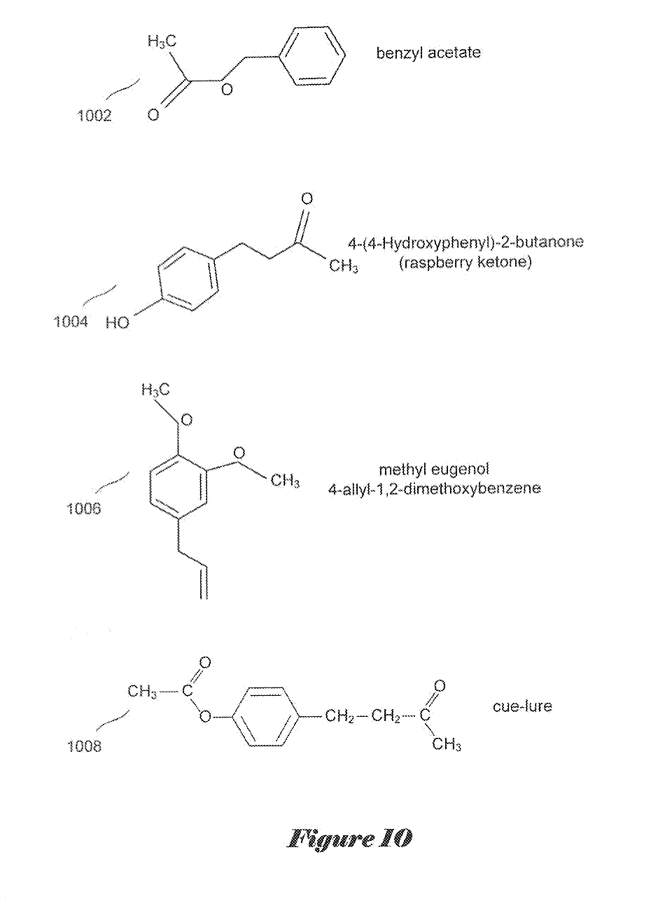

Components for multi-component insect-attracting substances that represent embodiments of the present invention may be selected based on a variety of different criteria, constraints, and parameters, as discussed above. In one embodiment of the present invention, a cost-effective multi-component insect-attractant substance targeting both oriental fruit flies and melon flies is desired. FIG. 10 illustrates four different, exemplary chemical attractants that may be employed to oriental fruit flies and melon flies. These attractants include benzyl acetate 1002, raspberry ketone 1004, and methyl eugenol 1006. A fourth attractant, cue lure, 1008, has been used alone as an insect attractant for the melon fly. Cue lure is essentially acetylated raspberry ketone, with an acetyl group added to the aryl hydroxyl group of raspberry ketone. Alternatively, cue lure is an acetic-acid ester formed from acetic acid and raspberry ketone. In the environment, cue lure is hydrolyzed to produce raspberry ketone. Both cue lure and raspberry keytone suffer, as insect attractants, from low volatility. Benzyl acetate is quite volatile, with a melting point of −51° C., and methyl eugenol is reasonably volatile, with a melting point of −4° C. Raspberry ketone is far less volatile, with a melting point of 82° C. However, raspberry ketone is a very effective chemical attractant for male melon fruit flies. Male oriental fruit flies are strongly attracted to methyl eugenol. As discussed above, as stated in the research paper provided in Appendix B, various studies and trials have shown that a combination of cue lure, chemically similar to raspberry ketone, and methyl eugenol is not an effective attractant for oriental fruit flies and is not significantly better at attracting melon flies than cue lure alone. It was commonly understood in the insect-lure industry, therefore, that a combination of raspberry ketone and methyl eugenol would not be an effective insect attractant for oriental fruit flies and would be more expensive than, but no better than, cue lure for attracting melon flies. Surprisingly, the methyl-eugenol/raspberry-ketone multi-component insect attractant that represents one embodiment of the present invention is a far better attractant for both oriental fruit flies and melon flies than methyl eugenol, alone, for oriental fruit flies or cue lure, alone, for melon flies. This methyl-eugenol/raspberry-ketone multi-component insect attractant is not only better at attracting both of these types of flies, but enables a single trap, containing the multi-component insect attractant, to be used in place of oriental-fruit-fly specific traps and melon-fly specific traps. In general, for monitoring for the presence of both oriental fruit flies and melon flies, using 50% percent or fewer traps that are more effective for attracting and luring both oriental fruit flies and melon flies than corresponding single-attractant traps represents a significant cost savings. Less traps need to be used, resulting in a significant initial cost savings, and the costs of monitoring and maintaining deployed traps, which scale according to the number of traps deployed, are also significantly decreased. Until the time of filing of the parent application or the current application, no insect traps employed a combination of methyl eugenol and raspberry ketone, for good reason. Because of the types of studies discussed in the research papers provided in Appendix B, no one thought that this combination would be effective.

Although the present invention has been described in terms of particular embodiments, it is not intended that the invention be limited to these embodiments. Modifications within the spirit of the invention will be apparent to those skilled in the art. For example, although vaporization and mechanical dispersion of multi-component insect-attracting substances of the present invention have been discussed, additional methods for introducing a given multi-component insect-attracting-substance embodiment of the present invention may be possible, including. As discussed above, embodiments of the present invention are directed to multi-component insect-attracting substances comprising a wide variety of different chemical-attractant components, designed to meet various criteria and parameters and to target particular types, groups, or species of insects. Although flying insects are discussed in the above examples, multi-component insect-attracting-substances may also be diffused into moist surfaces to attract crawling insects, diffused into bodies of water to attract water-dwelling insects, and introduced into many different environments.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A multi-component insect-attracting substance comprising:
a first chemical attractant, methyl eugenol; and
a second chemical attractant, raspberry ketone, having a number of physical characteristics that are different from corresponding characteristics of methyl eugenol, including far a lower volatility, that produce a multi-component plume with multiple chemical-attractant gradients and component plumes that differ substantially from one another in volume and radius and that attract oriental fruit flies and melon flies with greater effectiveness than either methyl eugenol or raspberry ketone, used alone.

2. The multi-component insect-attracting substance of claim 1 further including additional insect attractants, such as benzyl acetate.

3. The multi-component insect-attracting substance of claim 1 further including additional solvents and other chemical substances.

4. The multi-component insect-attracting substance of claim 1 deployed within a solid, timed-release lure comprising:
a fiberglass solid support;
the multi-component insect-attracting substance adsorbed to the fiberglass solid support; and
a polymeric matrix that encases the fiberglass solid support and multi-component insect-attracting substance to provide for timed release of the multi-component insect-attracting substance.

5. The multi-component insect-attracting substance of claim 1 deployed within a lure comprising:
a cotton wick; and
the multi-component insect-attracting substance adsorbed to the cotton wick.

6. The multi-component insect-attracting substance of claim 1 deployed within a lure comprising:
a porous lure body; and
the multi-component insect-attracting substance of claim 1 adsorbed to the porous lure body.
